(19) United States
(12) Patent Application Publication
(10) Pub. No.: US 2024/0177862 A1
(43) Pub. Date: May 30, 2024

(54) SYSTEM AND COMPUTER IMPLEMENTED METHOD FOR ASSISTED MANAGEMENT OF DECOMPENSATED CIRRHOSIS

(71) Applicant: CYBERLIVER LIMITED, Stanmore (GB)

(72) Inventors: Kalkivayi Seshagiri RAVI KUMAR, Manchester (GB); Rajeshwar Prosad MOOKERJEE, Northwood (GB)

(21) Appl. No.: 18/551,769

(22) PCT Filed: Mar. 22, 2022
(86) PCT No.: PCT/IB2022/052618

§ 371 Date: Sep. 21, 2023

Related U.S. Application Data
(60) Provisional application No. 63/165,052, filed on Mar. 23, 2021.

(51) Int. Cl. G16H 50/20; A61B 5/00; A61B 5/16; G16H 10/60; G16H 40/67

(52) U.S. Cl. CPC G16H 50/20; A61B 5/16; A61B 5/4244; A61B 5/746; A61B 5/7475; G16H 10/60; G16H 40/67

(57) ABSTRACT

The invention relates to a system for assessing decompensated cirrhosis (10). characterised by comprising: at least one patient application module configured to be connected to at least one sensor device (S) and/or at least one wearable device (W. B) to collect first patient data therefrom, and/or configured to receive a manual input of second patent data and/or configured to assess higher mental functions of a patient as third patient data: at least one practitioner application module configured to receive the first patient data and/or the second patient data and/or third patient data, and process the said patient data to detect signs related to decompensated cirrhosis complications. The invention further relates to a kit and a computer implemented method based on that system (10).

### FIELD OF INVENTION

The present invention relates to a system and computer implemented method for assisted diagnosis of new complications of decompensated cirrhosis, as well as enabling delivery of interventions - digital therapies- in this regard.

### BACKGROUND TO THE INVENTION

Patients with decompensated cirrhosis, defined as the development of ascites, hepatic encephalopathy (HE), gastrointestinal bleeding or bacterial infection, are at a high risk of new acute decompensation (AD) events and re-hospitalization.[1, 2] The readmission rate for patients discharged after an admission for AD remains as high as 40% [3], warranting carly outpatient follow-up, ideally every 2 to 4 weeks, in order to detect possible new complications and guide appropriate management. However, growing cirrhosis prevalence and over-burdened state healthcare systems, cannot facilitate such intensity of follow-up, currently further challenged by the pressures exerted by the COVID-19 pandemic.[5] Furthermore, cirrhosis patients in rural or deprived arcas may have limited access to specialist care, which negatively affects their outcomes.[6] Thus, there is an unmet need for tools to ensure timely, and equitable access to specialist liver care, to diagnose and manage new decompensation events, without the requirement for regular hospital reviews.

The current standard care pathway and treatment plan for patients with decompensated cirrhosis:
Patients admitted with decompensated cirrhosis are treated in hospital until stable and precipitating events of decompensation are controlled; they are then discharged home.
Upon discharge, they are provided with modified prescriptions, and limited advice on their nutrition and hydration.
Post discharge, patients are scheduled for clinical reviews in a specialist, hospital-delivered clinic. The initial follow-up is at 2-4 weeks, followed by a 6-8 week reviews, based on individual patient circumstances. If the condition of the patient becomes apparently stable, the review is extended to a 12-week review. During the clinical review, if the symptoms/signs have changed, they are advised a modified course of treatment accordingly.
In between the reviews, if the patients become unwell or develop new features of cirrhosis deterrioration, they often present urgently for re-hospitalization, with no community mechanism to intervene early and deliver specialist hepatology care.
Risks of hospital associated-infection and increased clinical service pressures, driving up time between clinical reviews, have increased the morbidity in cirrhotic patients.

Moreover, there are numerous problems in delivering care to decompensated cirrhotic patients:
Given the significant immuno-compromised nature of cirrhotic patients, they are at high risks of infection and need prompt treatment often with additional sepsis complications.
These patients require high levels of medical oversight. This has necessitated:
a. increased allocation of costs and hospital resources for clinical monitoring and treatment of decompensated cirrhotic patients, including intensive care;
b. increased clinician time due to the complex nature of their condition, and that current standard care pathway necessitates frequent out-patient direct clinical reviews, to ensure adequate assessment of their clinical state.
Decompensated cirrhotic patients require continuous, on-going care due to the nature of the illness. The time interval of scheduled hospital follow-up is 2-4 weeks for the initial review, followed by 6-12 weekly reviews, based on clinical indications of disease control. In time, there can be a very long gap between reviews:
a. Symptoms may worsen for patients, requiring re-hospitalization due to new, undiagnosed decompensation events.
b. COVID 19 and a growing liver disease burden have forced Hepatologists to rapidly implement telephone clinics to cope with demand for reviews. However, no clear tools exist to ensure cirrhotic patients receive timely and appropriate priority for interventions specific to their specific disease manifestations and severity, , nor mechanisms to capture clinical physical signs of cirrhosis complications remotely in the community.
There is no real-time, remote, at-home, safe monitoring and management system for decompensated cirrhosis.
a. This leads to zero real-time tracking and validation of patient regular vital signs, perceived wellbeing and associated precipitant factors, such as fluid intake, constipation, etc., between visits.
b. There is no immediate remote intervention available if new precipitants of cirrhosis complications or adverse clinical signs arise, and go unnoticed by patients through lack of regular, daily, continuous review systems.
Currently, there is a lack of availability of a simple, user friendly, low-cost, home based system for decompensated cirrhotic patient assessment, able to keep pace with the challenges of a dynamic clinical state such as decompensated cirrhosis.

### Description of innovation

Objectives of the present invention are to addresses these problems, including: lowering the risks of hospital-acquired infections in cirrhotic patients through reduced hospital exposure; limiting the costs and hospital resources in managing decompensated cirrhotic patients, providing regular, at-home, monitoring of relevant signs of cirrhosis complications using a user friendly, low-cost system, that also provides assisted patient management.

In order to achieve this objective, the invention relates to a system for assessing decompensated cirrhosis, comprising:
at least one patient application module configured to be connected to at least one sensor device and/or at least one wearable device to collect first patient data therefrom, and/or configured to receive a manual input of second patient data and/or configured to assess higher mental functions of a patient as third patient data;
at least one practitioner application module configured to receive the first patient data and/or the second patient data and/or third patient data, and process the said patient data to detect signs related to decompensated cirrhosis.

The signs detected may infer new decompensated cirrhosis events.

Advantageously, the system of the invention addresses the above problems by providing remote, real-time monitoring and clinical diagnostics, and personalized care within the safety of patients' homes. This achieves carly diagnosis of new complications, and early intervention when necessary, before the disease progresses, ideally avoiding hospital admissions or/and reducing the severity of presentations by up to 25-35% in a preferred embodiment.

According to other aspects taken individually, or combined according to any technically possible combination:
the said practitioner application module is configured to process the said patient data based on a processing database and processing algorithms for early detection and/or prevention of cirrhosis decompensation signs; and/or;
the at least one practitioner application module is configured to process the said patient data with the processing algorithms based on a combination of (a) standard deviation of event specific digital biomarkers values calculated over a period, (b) weightage of the event specific digital biomarker and (c) patient inputted data, to determine cirrhosis decompensation events including one or more of dehydration/acute kidney injury, new accumulation of ascites, infection and/or hepatic encephalopathy; and/or
the system comprises a patient application for a patient device, wherein the patient application comprises the said patient application module; and/or
the patient application is configured to provide nudges and/or reminders relating to detected signs related to decompensated cirrhosis, to a patient; and/or
the system comprises a practitioner application for a practitioner device, wherein the practitioner application comprises the said practitioner application module; and/or
the practitioner application is configured to provide or show to a practitioner, alerts relating to detected signs related to decompensated cirrhosis and/or interpretations of patient data implying signs related to decompensated cirrhosis; and/or
the said sensor device and/or wearable device is configured for an acquisition of patient data selected from the group comprising a temperature, heart rate, a heart-rate-variability, a blood pressure, an electrocardiogram, a physical activity, a sleep status, a weight, a body fluid composition, or a combination of at least two of those patient data; and/or
the said patient application module is configured to receive a manual input of patent data selected from the group comprising a food intake, a fluid intake, a reported sense of well-being (perceived well-being), or a combination of at least two of those patient data; and/or
the said patient application module is configured to assess a mental function of a patient through one or more mental function tests (higher mental function); and/or
the said wearable devices comprise a smart watch or other smart device and/or a blood pressure device; and/or
the said sensor devices comprise a bioimpedance smart scale and/or a thermometer; and/or
the system comprises a messaging application module, configured for correspondences between a patient and a practitioner.

The specific distinctive patient application module/device and wearables, the specific collected data, and the separate patient and practitioner devices, enable a monitoring of the patients that is precise and facilitates day-to-day measurements, and in real time. Moreover, the management system assists the clinician in the interpretation of the remotely acquired signs, albeit, as an expert, the clinician can always override the system's assessment to limit false positive interpretations.

The invention further relates to a kit for assessing decompensated cirrhosis, characterised by comprising
at least one patient application for a patient device, wherein the patient application comprises a patient application module configured to be connected to at least one sensor device and/or at least one wearable device to collect first patient data therefrom, and/or configured to receive a manual input of second patent data and/or configured to assess a mental function of a patient as third patient data; and
at least one practitioner application module configured to receive the first patient data and/or the second patient data and/or third patient data, and process the said patient data to detect signs related to decompensated cirrhosis.

The kit further comprises any other part or element of the system of the invention.

The invention also concerns a computer program product comprising instructions which, when the program is executed by one or more computerised units, causes the said computerised unit to carry out steps for:
acquiring first patient data from a patient, in particular through at least one sensor device and/or at least one wearable device,
acquiring second patient data through a manual input, and/or third data relating to an assessed a mental function of a patient (higher mental function), in particular through at least one patient application module;
processing the first patient data and/or the second patient data and/or third patient data to detect signs related to decompensated cirrhosis, in particular through at least one practitioner application module.

The computer program product further causes the computerised unit to carry out any other computer implemented action of the system of the invention.

According to other aspects taken individually, or combined according to any technically possible combination:
the computer program product further causes the computerised unit to carry out a step for providing nudges and/or reminders relating to detected signs related to decompensated cirrhosis, to a patient; and/or
the computer program product further causes the computerised unit to carry out a step for providing or showing a practitioner, alerts relating to detected signs related to decompensated cirrhosis and/or interpretations of patient data implying signs related to decompensated cirrhosis; and/or
the computer program product further causes the computerised unit to carry out a step for acquiring patient data selected from the group comprising a heart rate, a heart-rate-variability, a blood pressure, an electrocardiogram, a physical activity, a sleep status, a weight, a body fluid composition, or a combination of at least two of those data; and/or
the computer program product further causes the computerised unit to carry out a step for receiving a manual input of patent data selected from the group comprising a food intake, a fluid intake, a perceived wellness (well-being), or a combination of at least two of those data; and/or
the computer program product further causes the computerised unit to carry out a step for assessing a mental function of a patient (higher mental function) through one or more mental function tests; and/or
the computer program product further causes the computerised unit to carry out a step for exchanging communication between a patient and a practitioner.

The invented system and computer implemented method demonstrated (a) 38% reduction in hospital admissions through carly detection and intervention compared to standard follow-up pathway (b) 69% reduction in hospital stay duration through carly detection and intervention than in a control group (c) early detection and intervention in 82% of events (d) 85% patient engagement and (e) improved liver disease severity scores (CLIF-AD and MELD scores) in a 20 patient clinical study.

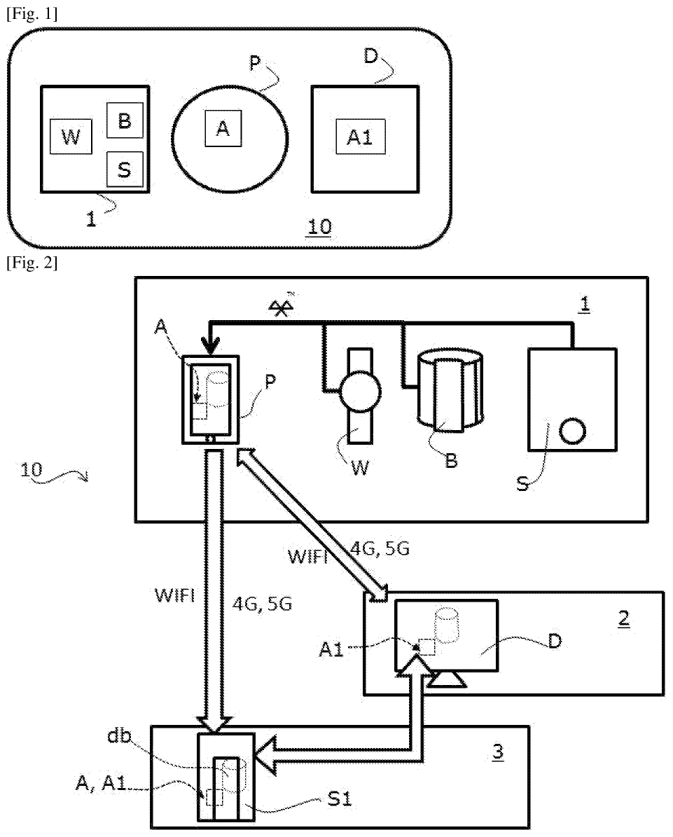

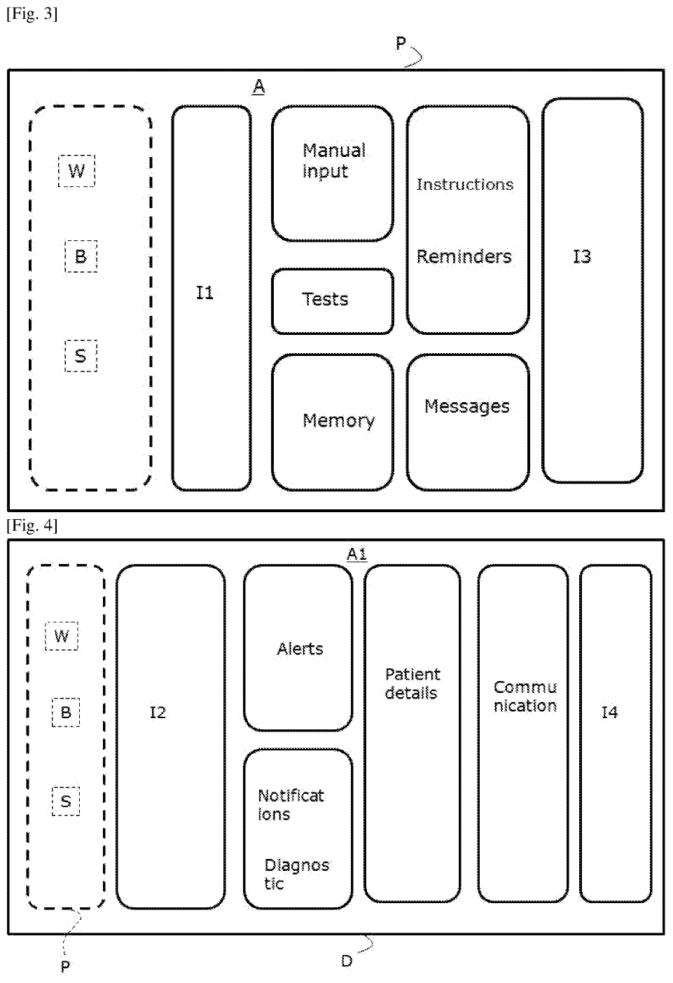

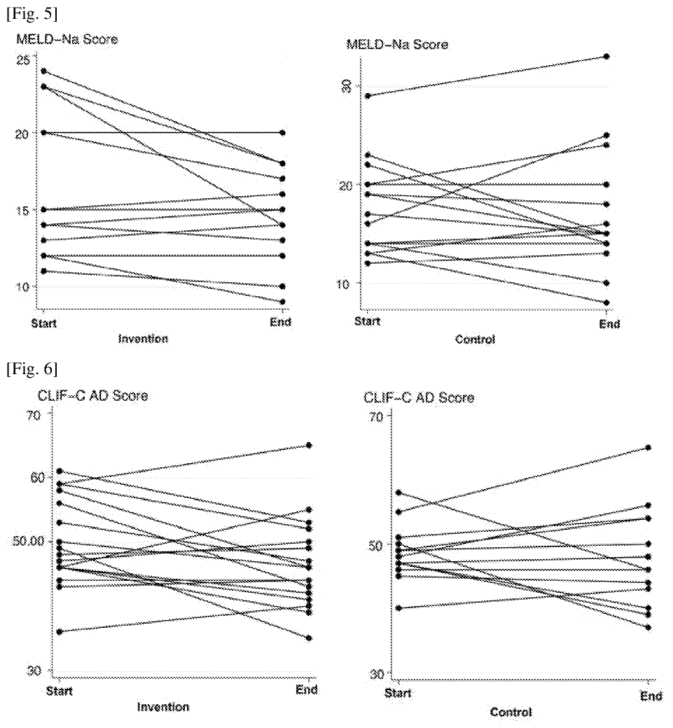

The invention will be further detailed by the description of non limitative embodiments, and based on the enclosed figures, among which:

[FIG. 1] is a general block diagram of an embodiment of the invention;

[FIG. 2] is a general scheme of a preferred embodiment of the invention;

[FIG. 3] is a block diagram of a patient application module of a system according to the preferred embodiment of the invention;

[FIG. 4] is a block diagram of a practitioner application module of a system according to the preferred embodiment of the invention;

[FIG. 5] illustrates results of tests based on scores at enrolment and end of a study, separately in patients managed with the invention and in controls patients, based on the MELD-Na Score; and

[FIG. 6] illustrates results of the same tests, but based on the CLIF-C AD Score.

The invention concerns a system 10 for assessing decompensated cirrhosis. The system 10 is a computer implemented system enabling a remote monitoring and a remote assessment of decompensated cirrhosis. Reference 1 relates for example to a patient home 1.

The system 10 may comprise at least one wearable device configured for an acquisition of first patient data. The wearable device may be a smart watch W (or other hardware collecting ECG analysis) and/or a smart blood pressure device B. Additional wearable devices may be used.

Alternatively or preferably in combination, the system 10 may comprise at least one sensor device configured for an acquisition of first patient data. The sensor device may be a bioimpedance smart scale S and/or a digital thermometer. Additional sensor devices may be used.

The system is preferably using generic wearable and sensor devices connected to the relevant application module.

The preference goes to wearable and sensor devices avoiding invasive acquisitions of the patient data, but enabling regular or constant sensing of the patient data. In particular, the patient may use normally a smart watch W (or other smart device) and his telephone, without needing to patch sensors to sense second patient data.

The terms “first patient data” should be interpreted as the patient data coming from the sensor device or the wearable device. A specific interface Il may be provided. The data sensed and collected are preferably transferred to another part of the system 10 for further processing.

The wearable device W or sensor device S is configured for acquiring patient data related to cirrhosis. These patient data may include objective data such as patientspecific metrics. The invention preferably implies also assessing subjective data, concerning the current self-reported state or well-being of the patient. In the preferred embodiment, the patient data may be one or more of a temperature, a heart rate, a heart-rate-variability, a blood pressure, an electrocardiogram (ECG), a physical activity, a sleep status, a weight, a body fluid composition, or a combination of at least two of those patient data.

In particular, the smart watch W (or other smart device) captures ECG, sleep and physical movements (such as steps); the blood pressure monitor B captures the blood pressure; the bio-impedance scale S captures the weight, body fluid and muscle mass compositions.

The system 10 further comprises at least one patient application module. The patient application module is part of a computer implemented application A to run in a computerized unit, such as smartphone application. In particular, the system 10 comprises a patient application A for a patient device P such as a smartphone of a patient. The patient application A comprises the said patient application module.

The patient application module is configured to be connected to the said sensor device S and/or wearable device W to collect the first patient data therefrom. Advantageously, this enables the use of simple wearable and sensor devices with a short-distance communication system such as a Bluetooth™M system, without further processing calculations in the wearable device W or sensor device S.

Alternatively or preferably in combination, the patient application module is configured to receive a manual input of second patent data. The terms “secondpatient data” should be interpreted as the patient data coming from the manual input in the patient application module. The data entered and collected are preferably transferred to another part of the system 10 for further processing. The manual input enables considerations relating to non-sensing variables and/or activity of the patient. In particular, the second data may be one or more of a food intake, a fluid intake, a perceived sense of wellbeing, or a combination of at least two of these patient data. The fluid intake may include water, juices or other non-alcoholic beverages, and/or alcoholic beverages. The perceived wellness may be entered using a scale of well being.

Alternatively or preferably in combination, the patient application module is configured to assess higher mental functions of a patient as third patient data. The terms “thirdpatient data” should be interpreted as the patient data coming from the assessment of the mental function in the patient application module. In particular, this assessment comprises providing one or more higher mental function tests, and collecting the corresponding data. Advantageously, this enables a simple remote assessment of impaired higher mental functions related to cirrhosis, termed hepatic encephalopathy. The patient application A may comprise a specific interface for these tests.

The system 10 further comprises at least one practitioner application module. The practitioner application module is part of a computer implemented application Al to run in a computerized unit. In the invention, the computerized unit for the practitioner application module is preferably a different unit from that of the patient application module. Thus, the diagnosis and interpretation of data or signs may be made or confirmed by a practitioner, such as a clinician, using the practitioner application module, for example in a computer D. The practitioner application Al comprises the said practitioner application module. The practitioner application Al and unit D are preferably in a clinical delivery or hospital site 2 different from the patient's home 1.

The invention may be applied to one or more practitioners, with multiple patients of the same practitioner; or sets of patients, cach set being followed-up by a specific practitioner.

The practitioner application module is configured to receive the patient data from the said wearable device W and/or sensor device S, and/or from the patient application module, and process the patient data to detect signs related to decompensated cirrhosis. A specific interface 12 may be provided in this regard. In the preferred embodiment, the first patient data are transmitted from the said wearable device W and/or sensor device S to the patient application module, then the first, second and third patient data are transmitted to the practitioner application module.

The system 10 preferably comprises a platform SI based for example on one or more servers for a preferred centralized storage and processing of the patient data to communicate to the practitioner application module. The servers and platform are preferably at a server site 3 different from the patient's home 1 and the hospital 2. Specific interfaces 13 and 14 may be provided to connect the platform to the patient application A and to the practitioner application Al respectively.

Advantageously, the system 10 of the invention enables lowering the risks of hospital-acquired infections in cirrhotic patients through reduced hospital exposure, limiting the cost and hospital resources in the management of cirrhotic patients, providing continuous, at-home 1, safe monitoring of relevant signs of cirrhosis complications using a user friendly, low-cost system, that also provides assisted patient management. The invention includes a multimodal monitoring system designed for decompensated cirrhotic patients with help of an automated, real-time software platform S1.

Depending on the embodiment to consider, the patient and practitioner applications A, Al may be partially or totally run directly from the platform SI only; and displayed in the practitioner device D and patient device P.

According to an embodiment, the said practitioner application module is configured to process the said patient data based on a processing database db, with proprietary algorithms, so as to facilitate an alert, when appropriate, of new decompensation events. In particular, the patient data/metrics are recorded through sensors/application and fed into the platform S1 in real-time. The platform S1 (or practitioner application module) process the data with CirrhoCare's algorithms [unique combination of (a) standard deviation of event specific digital biomarkers values calculated over a period, (b) weightage of the event specific digital biomarker and (c) patient inputted data] using a processing database db (to obtain a diagnosis or pre-diagnosis, prepare or alert on interventions and provide a preventive treatment care based inter allia on patient metrics for early detection and prevention of cirrhosis decompensation events.

In particular, the patient data/metrics are recorded through sensors/application and fed into the platform SI in real-time. The platform SI (or practitioner application module) process the data with CirrhoCare's algorithms [unique combination of (a) standard deviation of event specific digital biomarkers values calculated over a period, (b) weightage of the event specific digital biomarker and (c) patient inputted data ] using a processing database db (to obtain a diagnosis or pre-diagnosis, prepare or alert on interventions and provide a preventive treatment care based inter allia on patient metrics for carly detection and prevention of cirrhosis decompensation events.

These data may for example include statistics on the signs to be detected or the test results. Advantageously, the invention uses clinically validated facts for an improved accuracy of the interpretation of the patient data. More particularly, the platform S1 is a clinical diagnostic and therapeutic platform specifically designed by a team including hepatologists, for cirrhosis management, drawing on evidence based research and knowledge from clinical trials. The platform may offer the following.
a. Collation and integration of the information flow between the sensors, wearables, patient application A and clinical user dashboard D (i.c., practitioner application A1).
b. Clinically validated, assisted-diagnostic, coded algorithm, for diagnosing and providing carly interventions based on information captured by the application and that presented to the clinicians on the clinical dashboard D, described. The clinical diagnostic algorithm of the cirrhosis platform S1 uses a coded algorithm to facilitate early detection and intervention based on the nature of the specific and patient-personalised decompensation event.

According to an embodiment, the patient application A is configured to provide alerts and/or reminders relating to detected signs related to decompensated cirrhosis, to a patient.

This embodiment enables to provide timely interventions and reminders that include patient education and directed management. Moreover, based on patient inputs, the patient application module prompts patients for further action to provide further information or appropriate interventions for decompensated cirrhosis events.

According to an embodiment, the practitioner application Al is configured to provide or show to a practitioner, alerts relating to detected signs related to decompensated cirrhosis and/or interpretations of patient data implying signs related to decompensated cirrhosis. This embodiment enables an assisted-diagnostic practitioner application Al through a user dashboard D. Advantageously, this embodiment helps clinicians to view and monitor all relevant patients' metrics and information, in real-time. The information may be presented for assisted diagnosis and suggested potential treatments and interventions.

According to an embodiment, the system 10 comprises a messaging application module. The messaging application module is configured for communication between a patient and a practitioner. This may include audio, video, text messages or a combination of at least two of these media. This embodiment enables a direct intervention from or recommendations by the clinician, or reporting from the patient, for example of a current state of well-being, effects of a treatment or a recommendation.

This embodiment provides an ongoing two-way communication with a clinician, including for example voice messages, text messages or chats.

The preferred embodiment of the invention is a single integrated remote monitoring care management solution for cirrhotic patients that will primarily provide continuous, real-time remote monitoring. The solution has three inter-related elements to it:
1. the sensor and wearable devices for remote acquisition of important vital signs such as heart rate, heart-rate-variability, blood pressure, in addition to physical activity and sleep status, weight and body fluid composition.
2. the smartphone application A that captures higher mental function, helps patients input food, fluid, and perceived wellbeing status, and other data; automatically integrating the metrics from the sensor and wearable devices and also facilitating on-going communication between the clinician and patient.
3. the clinical user dashboard D that efficiently collates, organises, and presents patient data in a format that allows early detection of signs of cirrhosis complications, supports a diagnostic algorithm, and rapidly proposes a triage of patients who need direct clinical review, or alternative interventions.

The system 10 of the preferred embodiment is a pioncer, digital prescription therapeutic solution for cirrhosis by offering safe, at-home 1, remote monitoring and intervention for decompensated cirrhotic patients. The solution is managed by the platform SI and has preferably all of the components listed above.

In the preferred embodiment, the patient application A acts as a bi-directional communication channel between the clinician and the patient, in combination with the practitioner application A1. The application delivers interventions and reminders from the clinician and the platform SI to the patient. The practitioner application Al (clinical user dashboard D) is preferably for use by specialist Hepatology clinical staff. The clinical user dashboard D connects to the patient application A preferably via the platform SI interface.

In the preferred embodiment, the clinical user dashboard D is designed to direct clinicians to those patients with critical alerts based on colour codes (‘traffic-light’ system for example), helping triage those needing immediate patient advice and direct clinical review. The clinical dashboard D acts as an assisted-diagnostic system to indicate to clinicians, changes in the patient's metrics, or patient symptoms, which may indicate possible new decompensated cirrhosis events, and guiding carly intervention.

In addition, the invention enables an assisted-diagnosis method for use by specialist Hepatology clinical staff presented on the clinical user dashboard D for real-time monitoring and providing immediate interventions to treat or prevent progression from decompensation events.

The dashboard D is clinician-friendly, presenting clinicians with organised and colour-coded prioritisation of patient daily data, changes in metrics and patient symptoms, as well as suggested clinical interventions and patient communication channel.

The dashboard D (practitioner application A1) may implement one or more of the following actions: assists the clinician with real-time alerts when the patient metrics change, when the patient records symptoms indicating possible decompensation events, when the patients fail to take measurements or record information in-spite of automated reminders, and/or when the patient wants to communicate with the clinician, and give possible intervention recommendations. The dashboard D is designed to give clinicians information and direct triaging of critical patients based on colour codes (red, amber, green for example), to provide immediate patient advice and direct clinical review.

The patient application A may do one or more of the following, for example on an everyday basis: provide instruction based guidelines to capture vital signs and other metrics via sensor devices and wearable devices; capture higher mental functions, capture patient information by simple, easy to understand questions, provide patients with direct, ongoing voice-based and text-based communication with the clinicians; provides patients with clinician instructions and advice; provide patients with reminders and suggested clinical interventions.

The invention further relates to a kit for assessing decompensated cirrhosis, comprising any of the elements or parts of a system 10 as described above.

In particular, the kit comprises:
at least one patient application A for a patient device P, wherein the patient application A comprises a patient application module configured to be connected to at least one sensor device and/or at least one wearable device to collect the first patient data therefrom, and/or configured to receive a manual input of second patent data and/or configured to assess higher mental functions of a patient as third patient data; and
at least one practitioner application module configured to receive the first patient data and/or the second patient data and/or third patient data, and process the said patient data to detect signs related to decompensated cirrhosis.

The invention also comprises a computer program product A and/or Al comprising instructions which, when the program is executed by one or more computerised unit P, D, and/or S1, causes the said computerised unit to carry out any computer implemented action of the system 10 of the invention.

In particular, the computer program product further causes the said computerised unit to carry out steps for
acquiring first patient data from a patient, in particular through at least one sensor device S and/or at least one wearable device W, B,
acquiring second patient data through a manual input, and/or third data relating to an assessed mental function of a patient, in particular through at least one patient application module;
processing the first patient data and/or the second patient data and/or third patient data to detect signs related to decompensated cirrhosis, in particular through at least one practitioner application module.

Technological advancements and safe means to deliver quality care has changed the way healthcare works and the invention is the first step in delivering safe, at-home 1, continuous monitoring and care of patients with decompensated cirrhosis. Advantages of using the invention as a remote monitoring and clinical management system 10 for Cirrhotic patients are listed below:
Quality liver care delivered at home to patients with decompensated cirrhosis.
Daily tracking and monitoring of patient vitals, mental function, food and fluid intake and perceived well-being, which gives an advantage over the standard care of hospital out-patient, intermittent reviews.
Increase in remote access to patient care without the fear of hospital visits and cross infection.
Provide care independent of patient and clinician schedules.
Provide care in the privacy and safety of patient's home environment.
Easily accessible through patient's own sensor devices S, wearable devices W and smartphone devices P.
A digital solution that generates actionable, real world insights, that enables intelligent data-driven care management and clinical decision making.
Continuous, on-going voice based and text based communication between patient and clinicians.
Reduce clinicians' time by offering assisted-diagnostic and therapeutic solutions to improve care outcomes and prevent disease progression.
Improvement in everyday quality of patient life.
Ongoing reminders to help and empower patients to manage some self-care.
Early direct clinical reviews based on the data and analysis presented clearly on the clinical user dashboard D.

1Immediate alert systems to clinicians based on changes in patient metrics and wellbeing.
Ability to decrease admissions from cirrhosis decompensation.
Early interventions to prevent worsening of symptoms and progression of disease and reduce the number of cirrhosis complication events, and thereby preventing some hospitalizations.
Reduction in mortality through more personalised, equitable and standardised care.

### EXAMPLES

Study:

A clinical study for CirrhoCare was conducted at the Royal Free Hospital, London by including 20 patients with decompensated cirrhosis for remote management and followed up, and compared with 20 patients managed with standard care.

All patients were issued and educated to the use the CirrhoCare ™ App, developed by CyberLiver Ltd., along with integrated CyberLiver Animal Recognition Test (CL-ART) for cognitive dysfunction through analysis of reaction time and competency during the test on a supplied study-specific smartphone (including a SIM-card) and the connected monitoring devices through Bluetooth, commercially sourced, included: 1) a wristwatch (Withings Move), for determination of heart rate and movement 2) A blood pressure (BP) cuff (Withings BPM Connect); Three automatic, consecutive measurements by the cuff were taken and averaged 3) A weighing scales (Withings Body+) with bioimpedance to record weight and body-water percentage 4) thermometer.

Clinicians were trained and given access to the clinician dashboard that is designed to give clinicians quick access to critical patients based on color codes to provide immediate patient advice and direct clinical review. The clinical dashboard acts as an assisted-diagnostic system to indicate to clinicians changes in the patient's metrics, or patient symptoms, which may indicate possible decompensated cirrhosis events, and guiding early intervention. This mechanism would equate to performing a regular ward round in hospital, when the patient is at home, so as to action changes in patient status early, before a more severe complication arises.

### Study Findings—Technical

1. Patients received prompts/nudges/reminders every morning at an agreed time and were guided to successfully measure (a) ECG reading using the wristwatch, (b) blood pressure using the cuff, (c) weight and body-water percentage using weighing scale, and (d) take the timed cognitive function test using CL-ART more than once if unsuccessful and (e) input manually their sense of well-being (in order from well to unwell: ‘Fresh, Active, Well, Bit Tired, Dizzy, Weak’).
2. Patients received prompts/nudges/reminders every evening at 8 pm to manually input their fluid (amount and type of fluid, including a separate drop-down menu for alcohol beverages) and food (number of cooked and total meals) intake.
3. Clinicians were presented via the clinician dashboard with the clinical meaningful information such as monitoring and diagnostic alerts related to decompensated events with evidence and recommendation for review and intervention. Examples
   a. Clinician was presented with an alert highlighting the detection of possible ascites for patient-1 and upon review, Clinician promptly communicated patient to reduce the fluid intake, and increase diuretic does, averting the occurrence of the event.
   b. Clinician was presented with an alert highlighting the detection of possible Hepatic Encephalopathy for patient-3, in the context of recent nausea and vomiting; upon review, Clinician advised the patient to increase the fluid intake to 2000 ml/day and asked her to message in case she failed to improve. Patient was fine by 9 pm and communicated same to the clinician. If not for the intervention, patient would have continued to deteriorate.
   c. Clinician was presented with the patient-9′s self-reported well-being fluctuated between “Bit tired”, “Dizzy” and “Weak”, compared to “Well” the week before for 5 days (days 9-14 after inclusion),. There were no apparent changes in haemodynamic measures, CL-ART test, weight or body water percentage. When contacted, the patient informed the team that he had had diarrhoea in the previous week, and had felt weak and uncomfortable, but not confused. The patient was given advice on the importance of fluid intake of about 2000 ml/day to avoid dehydration and encouraged to continue his monitoring. He was also advised to hold Lactulose as long as diarrhoea persisted.

4. Clinicians and Patients exchanged communication by text/voice messages on actions to prevent complications related to decompensated cirrhosis to the patients.

### Study Findings—Clincial

1. 75% percent of the remotely managed patients showed excellent compliance with daily monitoring, which exceeded the reported adherence to pharmacological therapies in patients with advanced cirrhosis, of around 50% (7,8). A further 10% had good compliance, defined as performing at least 2-4 daily measurements per week.
2. On 16 occasions in 12 different patients, Investigators contacted patients due to CirrhoCare™ system clinical alerts based on measurements and selfreported data, for changes they deemed not severe enough to require hospitalization but felt might benefit from community-intervention, aiming to prevent re-admission. The following events were registered: 5 significant fluid accumulation, 2 carly HE (triggers: 1 dehydration and 1 constipation), 6 dehydration (risk of AKI), 2 cases of alcohol relapse and 1 general malaise due to a probable viral infection. The median time to first registered event was 18 (IQR 4-26) days. In these instances, after confirmation of clinical suspicion and repeat measurements, interventions such as advice on fluid intake, adjustment of diuretic dose or changing laxatives were implemented, guided by the CirrhoCare™ system data, as relevant. Alerts were mainly based on individual deviations in cognitive testing, BP, HR and weight/hydration status.
3. CyberLiver's patients had 38% fewer readmissions compared with contemporancous-control patients observed in parallel; the length of hospital stay upon readmission were also shorter, including the length of stay in intensive care. Furthermore, they had markedly fewer unplanned paracentesis requirements and most importantly, a greater improvement in MELD-Na Score (Model for End-Stage Liver Disease—sodium score) ([FiG. 5]) and CLIF-C Score (CLIF Consortium Acute Decompensation) ([FIG. 6]) than the control cohort

### References

[1] Jalan R, Pavesi M, Saliba F, Amoros A, Fernandez J, Holland-Fischer P, et al. The CLIF Consortium Acute Decompensation score (CLIF-C ADs) for prognosis of hospitalised cirrhotic patients without acute-on-chronic liver failure. J Hepatol 2015;62:831-840.
[2] Moreau R, Jalan R, Gines P, Pavesi M, Angeli P, Cordoba J, et al. Acute

on-chronic liver failure is a distinct syndrome that develops in patients with acute decompensation of cirrhosis. Gastroenterology 2013; 144:1426-1437, 1437 e1421-1429.
[3] Bajaj JS, Reddy KR, Tandon P, Wong F, Kamath PS, Garcia-Tsao G, et al. The 3-month readmission rate remains unacceptably high in a large North American cohort of patients with cirrhosis. Hepatology 2016;64:200-208.
[4] European Association for the Study of the Liver. Electronic address eee, European Association for the Study of the L. EASL Clinical Practice Guidelines for the management of patients with decompensated cirrhosis. J Hepatol 2018;69:406-460.
[5] Tapper EB, Asrani SK. The COVID-19 pandemic will have a long-lasting impact on the quality of cirrhosis care. J Hepatol 2020;73:441-445.
[6] Goldberg DS, Newcomb C, Gilroy R, Sahota G, Wallace AE, Lewis JD, et al. Increased Distance to a Liver Transplant Center Is Associated With Higher Mortality for Patients With Chronic Liver Failure. Clin Gastroenterol Hepatol 2017; 15:958-960.
[7] Hayward KL, Valery PC, Cottrell WN, Irvine KM, Horsfall LU, Tallis CJ, et al. Prevalence of medication discrepancies in patients with cirrhosis: a pilot study. BMC Gastroenterol 16:114. 2016.
[8] Polis S, Zang L, Mainali B, Pons R, Pavendranathan G, Zekry A, et al. Factors associated with medication adherence in patients living with cirrhosis. J Clin Nurs;25:204-212. 2016.

1. A system for assessing decompensated cirrhosis, characterised by comprising:
patient application modules for patient devices, the patient application modules being configured to be connected to sensor devices and/or wearable devices to collect first patient data therefrom from patients, and configured to receive a manual input of second patient data from patients and configured to assess higher mental functions of patients as third patient data;
at least one practitioner application module configured to receive the first patient data and/or the second patient data and/or third patient data, and process the said patient data to detect signs related to decompensated cirrhosis.

2. The system according to claim 1, wherein the said practitioner application module is configured to process the said patient data based on a processing database and processing algorithms for early detection and/or prevention of cirrhosis decompensation signs.

3. The system according to claim 1, wherein the at least one practitioner application module is configured to process the said patient data with the processing algorithms based on a combination of (a) standard deviation of event specific digital biomarkers values calculated over a period, (b) weightage of the event specific digital biomarker and (c) patient input data, to determine cirrhosis decompensation events including one or more of dehydration/acute kidney injury, new accumulation of ascites, infection and/or hepatic encephalopathy.

4. The system according to claim 1, further comprising a patient application for a patient device, wherein the patient application comprises the said patient application module, or a messaging application module for communication between a patient and a practitioner and is configured to provide nudges and/or reminders relating to detected signs related to decompensated cirrhosis, to a patient.

5. The system according to claim 1, comprising a practitioner application for a practitioner device, wherein the practitioner application comprises the said practitioner application module and is configured to provide or show to a practitioner, alerts relating to detected signs related to decompensated cirrhosis and/or interpretations of patient data implying signs related to decompensated cirrhosis.

6. The system according to claim 1, wherein the said sensor device and/or wearable device is configured for an acquisition of patient data selected from the group comprising a temperature, a heart rate, a heart-rate-variability, a blood pressure, an electrocardiogram, a physical activity, a sleep status, a weight, a body fluid composition, or a combination of at least two of those patient data.

7. The system according to claim 1, wherein the said patient application module is configured to receive a manual input of patent data selected from the group comprising a food intake, a fluid intake, a perceived well-being, or a combination of at least two of those patient data.

8. The system according to claim 1, wherein the said patient application module is configured to assess a mental function of a patient through one or more higher mental function tests.

9. The system according to claim 1, wherein the said wearable and sensor devices comprise a smart watch and/or a blood pressure device and/or bioimpedance smart scale and/or a thermometer.

10. A kit for assessing decompensated cirrhosis, characterised by comprising:
patient applications for patient devices, wherein the patient application comprises a patient application module configured to be connected to at least one sensor device and/or at least one wearable device to collect first patient data therefrom, and configured to receive a manual input of second patent data and configured to assess a mental function of a patient as third patient data; and
at least one practitioner application module configured to receive the first patient data and/or the second patient data and/or third patient data, and process the said patient data to detect signs related to decompensated cirrhosis.

11. A computer program product comprising instructions which, when the program is executed by one or more computerised unit, causes the said computerised unit to carry out steps for
acquiring first patient data from patients, in particular through at least one sensor device and/or at least one wearable device,
acquiring second patient data through a manual input, and third data relating to an assessed mental function of a patient, in particular through patient application modules;
processing the first patient data and/or the second patient data and/or third patient data to detect signs related to decompensated cirrhosis, in particular through at least one practitioner application module.

12. The computer program product according to claim 11, further causing the computerised unit to carry out a step for providing nudges and/or reminders relating to detected signs related to decompensated cirrhosis, to a patient.

13. The computer program product according to claim 11, further causing the computerised unit to carry out a step for providing or showing a practitioner, alerts relating to detected signs related to decompensated cirrhosis and/or interpretations of patient data implying signs related to decompensated cirrhosis.

14. The cComputer program product according to claim 11, further causing the computerised unit to carry out a step for acquiring patient data selected from the group comprising a heart rate, a heart-rate-variability, a blood pressure, an electrocardiogram, a physical activity, a sleep status, a weight, a body fluid composition, or a combination of at least two of those data.

15. The computer program product according to claim 11, further causing the computerised unit to carry out a step for receiving a manual input of patent data selected from the group comprising a food intake, a fluid intake, a perceived well-being, or a combination of at least two of those data.

16. The computer program product according to claim 11, further causing the computerised unit to carry out a step for assessing higher mental functions of a patient through one or more mental function tests.

17. The computer program product according to claim 11, further causing the computerised unit to carry out a step for exchanging communication between a patient and a practitioner.
